(19) United States
(12) Patent Application Publication
(10) Pub. No.: US 2023/0346491 A1
(43) Pub. Date: Nov. 2, 2023

(54) SYSTEMS AND METHODS FOR EMBODIED ROBOT CONTROL

(71) Applicant: e1ectr0n, Inc., Portland, OR (US)

(72) Inventors: Jonathan R. Bohren, Brooklyn, NY (US); Spencer Topel, Brooklyn, NY (US)

(21) Appl. No.: 18/309,074

(22) Filed: Apr. 28, 2023

Related U.S. Application Data
(60) Provisional application No. 63/363,910, filed on Apr. 29, 2022.

(51) Int. Cl. A61B 34/30; A61B 34/00

(52) U.S. Cl. CPC A61B 34/30; A61B 34/70

(57) ABSTRACT

The disclosed automation systems multiple optical sensors to dynamically and arbitrarily position one or more manipulators to engage with a workpiece in a continuous and precise manner in a workspace. The manipulators can be precision robotic arm(s) or component(s) that engage with the workpieces, such as parts and tools. The optical sensors obtain real-time positioning data for the robotic arms or components to allow them to precisely engage with the workpieces and avoid collision with each other.

### CROSS-REFERENCE TO RELATED APPLICATIONS

This application claims priority and the benefit from the U.S. Provisional Patent Application 63/363,910, filed Apr. 29, 2022, and titled. “SYSTEMS AND METHODS FOR EMBODIED ROBOT CONTROL,” which is incorporated herein by reference in its entirety for all purposes.

### BACKGROUND

Robotic manipulators are deployed in a wide variety of manufacturing automation processes, for example aerospace, medical, electronics and automotive industries. In recent years the use of 3D sensor systems, such as passive and active stereo depth cameras, have become a standard means of determining the position and orientation of relevant materials for robotic manipulation and machine automation applications. There are predominantly two means of integration of such sensor systems: 1) Sensors mounted to immobile features that observe relevant materials in key portions of an application's workspace; 2) Sensors mounted to the robotic manipulator, which is handling the relevant materials, referred to as “eye-in-hand.” The first means of integration is sufficient for tasks with low precision requirements and precludes frequent changeover or modification to process. The second means of integration is used for tasks with high precision requirements but suffers from tool occlusions which prevent continuous sensing of the relevant materials.

High-precision and continuous 3D sensing becomes necessary in applications Where the position and orientation of materials is more uncertain due to looser fixturing, lower robot rigidity, variability in part characteristics, a high mixture of parts, or some other system change that reduces the predictability of the automated process.

However, in a dynamic environment, 3D sensing performance can be critically degraded due to aspects of the automated process. These might include non-visible or partially non-observable components due to a shift in some aspect of the workspace, lighting occlusion, poor sensing accuracy due to distance from the workpiece or poor alignment to the workpiece, or by occlusion of the materials from the sensor field of view, caused by any number of factors such as: human workers, shading of part features, other robotic arms, or the robotic arm itself. Additional sensor systems may be deployed to mitigate some of these issues, but doing so requires detailed non-recurring integration work as well as recurring costs due to increased system complexity, processing power, network resources, and configuration and calibration time.

In tasks using robotic manipulators with lower joint and link stiffness than traditional industrial robotic manipulators, or in tasks requiring greater accuracy and precision, such as those that are only currently feasibly performed by human workers, the positioning of the sensors in a 3D sensor system becomes a critical factor for estimating the pose of objects and robots using specialized equipment. Specifically, 3D sensor systems or camera vision systems with the ability to measure depth, which may include stereo depth cameras, time-of-flight sensor systems, and associated methods.

The art would benefit from a dynamic and arbitrary automation system with continuous and precise real-world positioning data that can engage with objects.

### BRIEF DESCRIPTION OF THE DRAWINGS

Non-limiting and non-exhaustive embodiments of the invention are described with reference to the following drawings. In the drawings, like reference numerals refer to like parts throughout the various figures, unless otherwise specified, wherein:

FIG. 1 Generalization of the Embodied Manipulator Control (EMC) System. This figure exemplifies the external inputs and outputs to the system.

FIG. 2 EMC Physical Decomposition. The physical components and interactions in the EMC as they relate to the external inputs and outputs.

FIG. 3 EMC Instruction Functional Architecture A detailed characterization of the instructional stages and process between human operator and EMC.

FIG. 4 EMC Execution Functional Architecture (Fine) A detailed characterization of the fine position execution functional architecture between a high-level controller and EMC.

FIG. 5 EMC Execution Functional Architecture (Coarse) A detailed characterization of the coarse position execution functional architecture between a high-level controller and EMC.

FIG. 6 shows another embodiment of the disclosed robot control systems that has multiple manipulators.

FIG. 7 is a task-level controller that controls a position of a sensor manipulator via a workpiece pose optimization planner.

### DETAILED DESCRIPTION

The subject matter of embodiments disclosed herein is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

Robustness: It is an object of this invention to enable precision robotic manipulation of parts and tools in a workspace relative to physical features that are located at poses that may differ from the poses specified in a program.

Versatility: It is an object of this invention to enable precision robotic manipulation of parts and tools to a plurality of poses within a workspace without having to modify the physical structure of the workspace.

Low Code: It is an object of this invention to enable a user to efficiently program a robotic manipulation job in such a way that the programming system minimizes the need to write computer code.

Remote: It is an object of this invention to enable a user to program a robotic manipulation job without touching robotic manipulators nor being physically present in the location where the robotic manipulation is being performed.

A workpiece includes multiples physical parts that are manipulated, assembled, inspected, or disassembled by the system. A task is a durative activity occurring over a finite time with a defined start state, end state, and constraints, which define relationships between plurality of parts and a plurality of parts comprising a workpiece. A robotic manipulation arm is a robotic arm which can mechanically interact with a plurality of workpieces. A robotic sensor arm is a robotic arm which can dynamically move a plurality of sensors in 3D space. A job is a collection of tasks to be performed on a workpiece with a plurality of robotic manipulation arms and a plurality of robotic sensor arms. A workspace is a volume comprising a plurality of workpieces, robotic manipulation arms, robotic sensor arms, sensors, and other physical hardware necessary to perform a job.

To achieve Object 0006 and Object 0007, the disclosed system has one or more robotic manipulation arms, one or more robotic sensor arms, and a plurality of computers which execute a coordinated control program to implement a method of dynamically positioning a plurality of sensors in a workspace to observe both workpieces and robotic manipulation arms. Through positioning a sensor arm and processing sensor data with a plurality of computers, Object 0006 is realized with vision-guided control of the robotic manipulation arms. Through re-positioning a sensor arm, Object 0007 is realized as the sensors can be moved dynamically for each task in a job.

To achieve Object 0008, the disclosed system has a computer interface Which implements a method where a human operator controls both a plurality of sensor arms and a plurality of manipulation arms in real-time to encode sequences of sensor arm viewpoints and manipulation arm tasks along with additional task details.

To achieve Object 0009, the disclosed system includes a networked computer system which enables a remote operator to perform Method 00012 without being physically present with the computer system described in System 00011.

Embodiments will be described more fully hereinafter with reference to the accompanying drawings, which form a part hereof, and which show, by way of illustration, exemplary embodiments by which the systems and methods described herein may be practiced. The systems and methods may, however, be embodied in many different forms and should not be construed as limited to the embodiments set forth herein; rather, these embodiments are provided so that this disclosure will satisfy the statutory requirements and convey the scope of the subject matter to those skilled in the art.

The main characteristics of the embodiments described hereinafter will be listed. The technical elements described hereinafter are each independent technical elements, exhibit technical usefulness on their own or in various combinations, and are not limited to the combinations described in the claims at the time of application.

In the disclosed embodied robot manipulation control systems, the operator can manipulate the robot manipulation arms with precise feedback through the sensor data acquired by the sensor payload as it observes the workpiece. This feedback is sufficient for the operator to remotely pilot the system without being physically present with the system.

Also in the disclosed embodied robot manipulation control systems, a fixed sensor payload may be disposed at the end-effector portion of a sensor manipulator. By disposing a fixed sensor payload on a sensor manipulator, the operator can pilot the sensor payload to an optimal distance from the workpiece independent of the position of the work manipulator. Specifically, optimal distance is defined here as the distance that maximizes the precision of the sensor, subject to visibility constraints on the relevant features on the workpiece and the work manipulator, and furthermore without interfering with the motion of the work manipulator or workpiece.

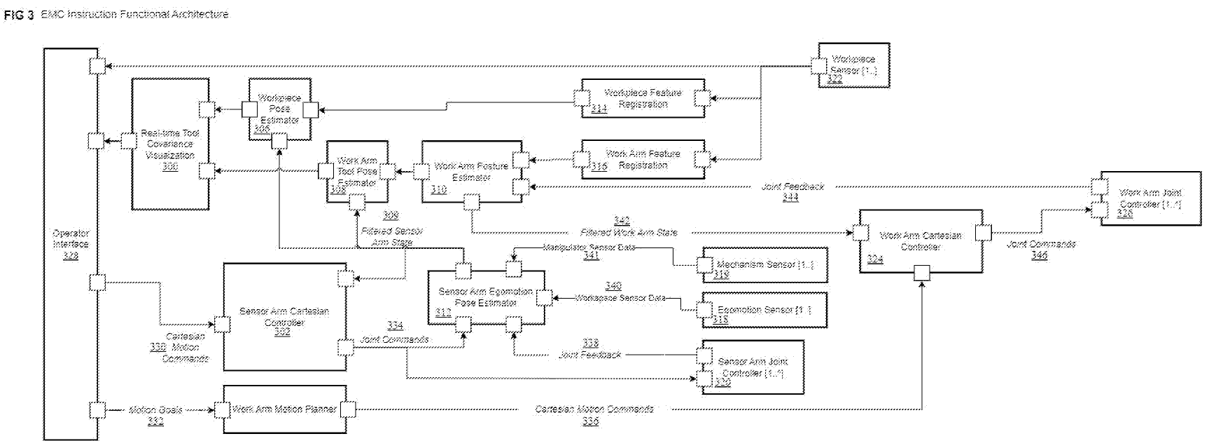

Even further, in the disclosed embodied robot manipulation control systems, an optimal sensing distance is defined as the distance that maximizes the precision of the sensor, subject to visibility constraints on the relevant features on the workpiece and the work manipulator. This optimal sensing distance allows the sensor manipulators to avoid interfering with the motion of the work manipulator or workpiece. The sensor manipulator can be positioned to this optimal sensing distance as detailed in FIG. 3 either automatically or as directed by the human operator.

Still further in the disclosed embodied robot manipulation control systems, disclosed in the present description, the operator provides sufficient instruction to the system so that it may perform the task or tasks like it autonomously without further human instruction.

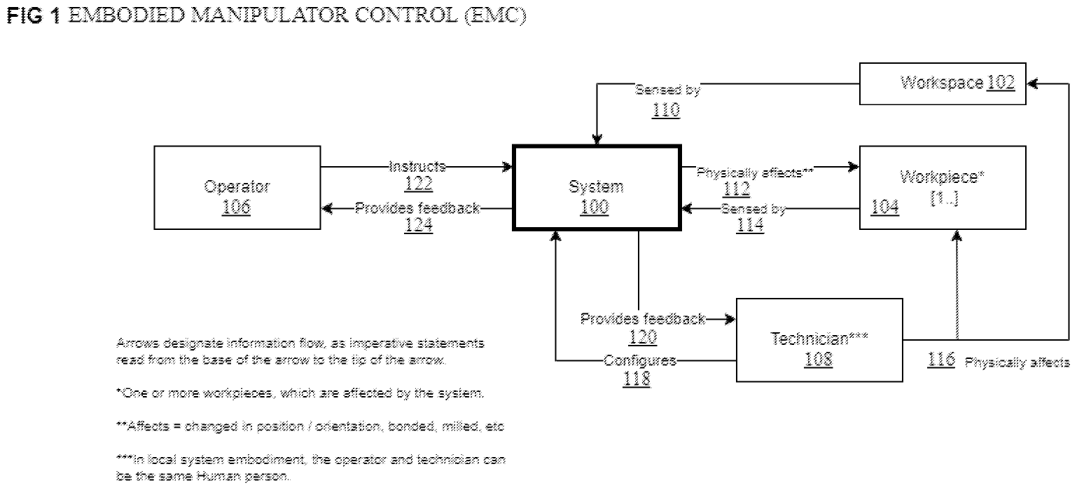

FIG. 1 shows an embodied manipulator control system that includes a robotic semi-autonomous system 100, which is employed in a configuration stage, programming stage, and execution stage. In all stages, system 100 senses 110 a workspace 102 using various optical sensors, and senses one or more workpieces 104.

In the programming and execution stages, system 100 physically affects 112 one or more workpieces 104 by positioning and engaging with the workpieces in various manners to manipulate the workpiece(s) that may include but is not limited to: screwdriving, interlocking, kitting, fixturing, bonding, milling, sanding, polishing, or some other manipulation requiring fine position control.

In the configuration stage of the system 100, a technician 108 (or other user) physically configures 118 the system 100 components using feedback 120 provided by System 100 through a technician console 222 and technician interface 224. The technician 108 then physically affects 116 the workpiece 104 and workspace 102, which may consist of calibration routines for system 100, placement of components for system 100, or placement of workpieces 104 or various support hardware or fixtures therein. In this embodiment, the technician 108 and the operator 106 roles may be performed by the same human worker.

In the instruction stage, this system 100 is controlled by a local or remote human operator 106 who instructs 122 the System 100 based on visual feedback 124 provided by the system 100 through an operator console 218.

The instructions provided by the human operator 106 include Cartesian motion commands, identification, and labelling of visual features in sensor data, motion constraints, and other annotations relevant to the task.

The visual feedback 124 provided by the system 100 includes both sensor data and summary data derived from the sensor data.

The result of the programming stage is a set of instructions encoding a task sequence which comprises the sensor manipulator motion, work manipulator motion, and other task-relevant information provided during the programming stage.

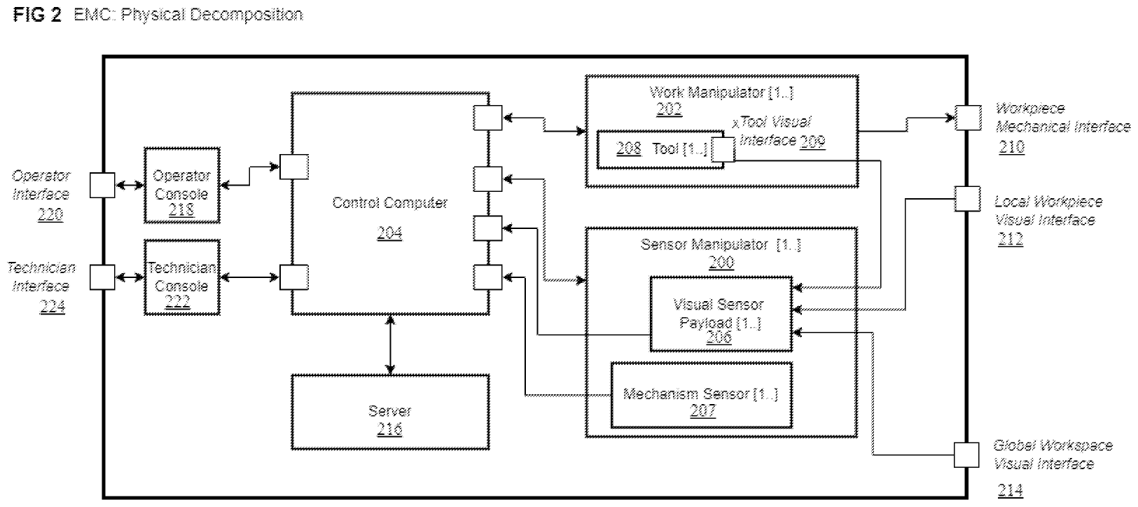

The embodied robot control system is an apparatus which performs tasks using a work manipulator 202 which is controlled by a control program running on a control computer 204 using feedback signals from a sensor payload 206 attached to a sensor manipulator 200 as illustrated in FIG. 2.

The sensor manipulator 200 consists of a mechatronic system which conveys a fixed sensor payload 206 in 3D space. The sensor payload 206 comprises one or more independent sensors which measure physical properties of the workpieces 104 in the workspace 102, and the physical properties of the work manipulator 202.

The sensor data collected by the sensor payload 206 of the sensor manipulator 200 is used to dynamically estimate the posture of the work manipulator 202, which is then used by the control program running on the control computer 204 to position the work manipulator 202 to perform the task upon a workpiece 210 using one or more tools 208.

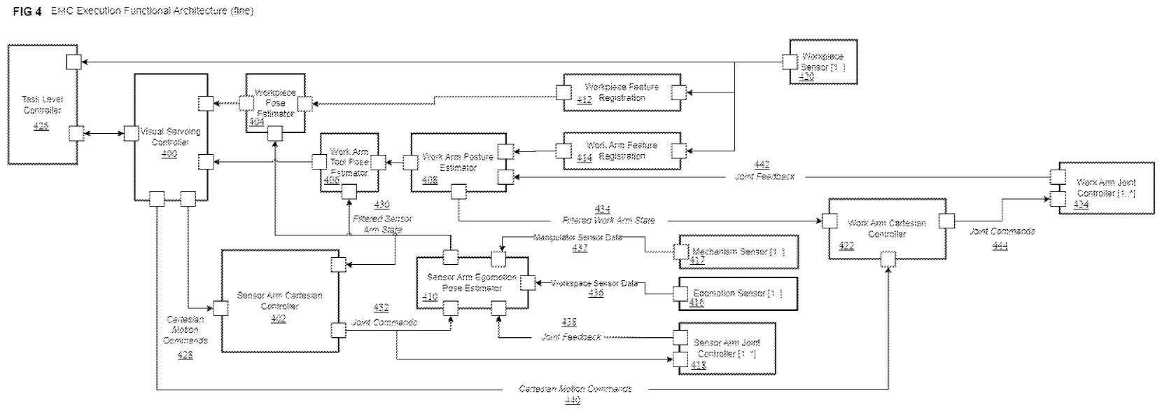

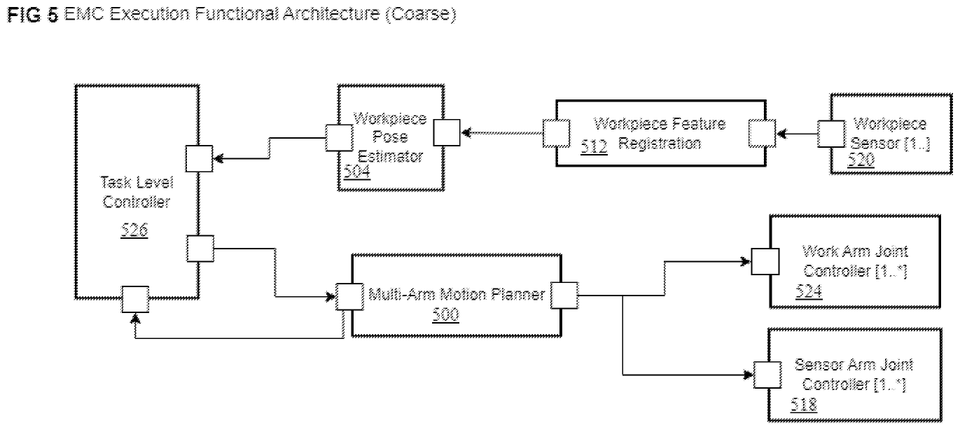

In the execution stage, positioning of the sensor manipulator 200 and work manipulator 202 is performed in two steps: 1) coarse positioning FIG. 5: whereby a sensor 214 consisting of a camera or other optical device locates and measures the pose of the workpiece 104 within the volume of the workspace 102, thereby allowing control computer 204 to compute the pose and path plan of the sensor manipulator 200 using either a server consisting of some remote or local computing resource 216, and 2) fine positioning FIG. 4: whereby the sensor manipulator 200 and work manipulator 202 are positioned through visual servoing based on the program generated by the programming stage.

The instruction functional architecture illustrated in FIG. 3 shows the communication between components during the instruction stage.

An operator via an operator interface 328 provides instructions in the form of and task-relevant information and Cartesian motion commands 330 to the sensor arm cartesian controller 302, and motion goals 332 to the work arm motion planner 304.

These instructions define goals which are sent to the work arm motion planner 304, which produces joint commands 346 which are transmitted to the work arm joint controller 326, which controls the work manipulator 202 and the tool 208 to physically affect 112 a workpiece 104 resulting in a performed task.

The Cartesian motion commands 330 are recorded by the control computer 204 or server 216 to build the instruction of the task operation executed by the task level controller 426.

The operator is guided based on information provided by a real-time tool covariance visualization 300 which illustrates the sensor precision at any given time.

The real-time tool covariance visualization 300, is generated by integrating data on the workpiece pose estimator function 306, processed workpiece feature registration function 314, and the work arm tool pose estimator function 308 through the posture estimator function 310, and the work arm tool pose estimator function 308 processed by the work arm feature registration function 316, all of which is captured from the workpiece sensor 322.

The execution functional architecture illustrated in FIG. 4 is detailed as follows: in the execution of an instructed program, the operator interface 328 is replaced with an interface to the task level controller 426, which queries the functional instructions from the control computer 204 or server 216 necessary to perform the instructed task illustrated in FIG. 3. Similarly, the real-time covariance visualization 300 is replaced with an interface to the visual servoing controller 400.

In both functional stages instruction and execution illustrated FIG. 3, FIG. 4, the estimated pose of the sensor payload 206 of the sensor manipulator 200 is computed by fusing internal mechanism sensors 207, 319, 417 and visual sensor 206, also referred to as egomotion sensors 318, 416, including optical sensors on the sensor manipulator 200 that observe the workspace and inertial sensors which observe mechanical motion, to correct for mechanical deflection in the sensor manipulator.

In both functional stages, instruction and execution illustrated FIG. 3, FIG. 4, the posture of the work manipulator 202 is estimated from the visual appearance of the work manipulator 202, by the work arm posture estimator 310 in the sensor data captured by the workpiece sensor 322 located on the sensor payload 206 on the sensor manipulator 200, to correct for mechanical deflection in the work manipulator 202.

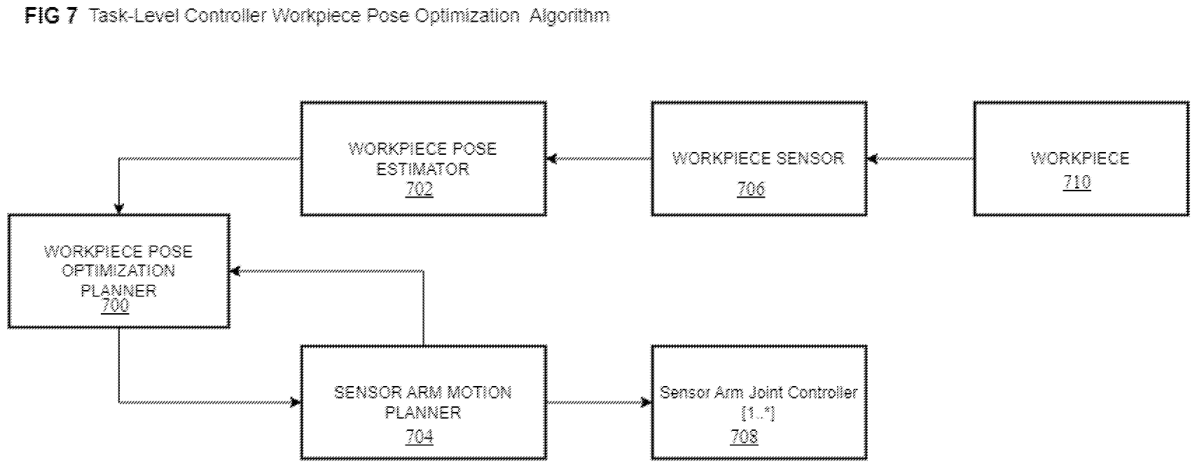

An aspect of this invention is the ability to move a position of the sensor manipulator. FIG. 7 illustrates how a task-level controller might make improvements to the sensor arm pose via a workpiece pose optimization planner 700. This algorithm would consider both the pose of the workpiece through the workpiece pose estimator 702 and the sensor arm motion planner 704. Using a feedback control loop, the workpiece optimization planner would arrive at an achievable pose for the sensor arm joint controller 708, which allows for the optimal position of the workpiece sensor 706 to make an optimal workpiece pose estimation 702. An example of this is optimization of a viewpoint that perceives the roundness of a screw hole, which would provide the system with better three-dimensional information by which to apply a screwdriving operation.

Another embodiment of the disclosed embodied robot control systems includes a multi-manipulator control system explained in the first embodiment as a manipulator control system and detailed in FIGS. 1-5.

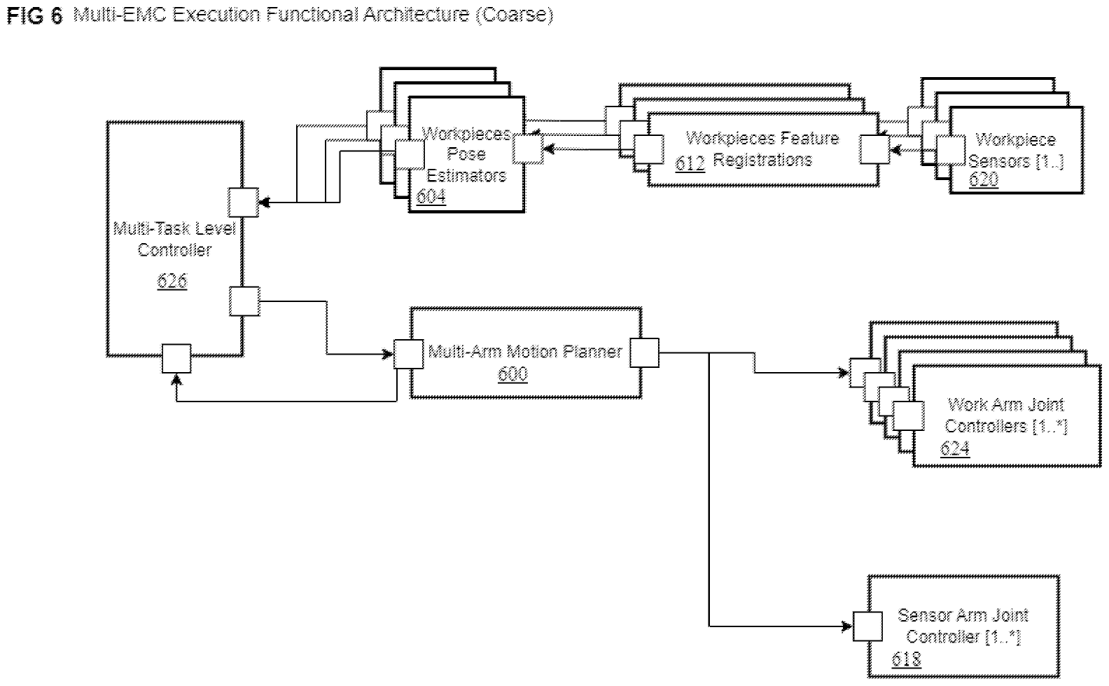

In this multi-manipulator control system embodiment, illustrated in FIG. 6, a primary sensor manipulator 618 and other workpiece sensors 620, that may be attached to the primary sensor manipulator or to fixtures around the workpiece, performs sensing to determine workpiece registration features 612, and from this, workpiece pose estimations 604. This information is transmitted to a multi-task level controller 626, that creates instructions to perform specific sequential or parallel tasks. This in turn is broken down into movement operations and collision estimations by the multi-arm motion planner 600, which then generates instructions sent to specific work arm joint manipulators 624, and a sensor arm joint manipulator 618. The need for such an embodiment should be relatable to human interaction with the workpiece. Generally speaking, more than one manipulator allows for a wider range of work tasks, such as holding or moving objects as the manipulators work on them, and to reorient an object for a more optimal position for manipulation.

The subject matter of embodiments disclosed herein is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

What is claimed is:

1. A system for manipulating a workpiece, comprising:
a sensor manipulator configured to sense a workpiece parameter or characteristic;
a sensor controller configured to control a position or function of the sensor manipulator;
a work manipulator separately moveable from the sensor manipulator and configured to engage with the workpiece; and
a work controller configured to:
receive data about the position or function of the sensor manipulator, and
generate an instruction that causes the work manipulator to take action to engage with the workpiece, the instruction based on the received position or function of the sensor manipulator.

2. The system of claim 1, wherein the sensor manipulator is located on a robotic arm that maneuvers around but does not engage with the workpiece.

3. The system of claim 1, wherein the sensor manipulator includes one or more optical sensors, and wherein at least one of the optical sensors is configured to sense the workpiece and the work manipulator, and another of the optical sensors is configured to estimate the position of the sensor manipulator.

4. The system of claim 3, wherein the optical sensor includes one or both of a camera or a combined light projection and emitting optical sensor.

5. The system of claim 1, wherein the workpiece parameter or characteristic includes one or more of a physical property, assembly features, or a configuration property.

6. The system of claim 1, wherein the workpiece parameter or characteristic includes one or more of shape, color, mate points, referenced dimensions, or a relationship between different workpieces.

7. The system of claim 1, wherein the sensor controller is further configured to be driven or piloted by a user during an instruction stage.

8. The system of claim 1, wherein the sensor controller is further configured to be driven by a motion planner during an execution stage, the motional planner driving the sensor controller based on goals recorded during an instruction stage relative to workpiece parameters.

9. The system of claim 1, wherein the sensor controller is discrete from the work controller.

10. The system of claim 9, wherein, during an execution stage, the sensor controller and the work controller are driven by a common coordinating controller, the common coordinating controller configured to use motion planning to achieve a point of view such that the workpiece features and the work manipulator features are visible to the sensor manipulator.

11. The system of claim 10, wherein common coordinating controller is configured to use visual servoing to both track the workpiece with the sensor manipulator and to achieve a work goal with the work manipulator.

12. The system of claim 1, wherein the work manipulator includes a robotic arm that is structured to maneuver to engage with the workpiece.

13. The system of claim 1, wherein the work manipulator includes multiple robotic arms that are structured to maneuver to engage with the workpiece, the work controller further configured to determine coordinate positions of each of the multiple robotic arms to allow each robotic arm to engage with the workpiece and to avoid collision with the other multiple robotic arms.

14. The system of claim 1, wherein the instruction generated by the work controller is an engagement instruction, and wherein the work controller is further configured to generate a position instruction that causes the work manipulator to translate the position or function of the sensor manipulator to a work manipulator responsive action.

15. The system of claim 1, wherein the work controller is further configured to generate a work path plan for the work manipulator to follow.

16. The system of claim 1, wherein the sensor manipulator includes an egomotion filter for the sensor manipulator, the egomotion filter configured to:
track a location of a tip of the sensor manipulator relative to a surrounding environment; and
correct for any structural deflection in the sensor manipulator.

17. The system of claim 1, wherein sensors on the sensor manipulator and hardware sensor on the work manipulator are configured to track a joint-space configuration of the work manipulator and adjust the work manipulator to a predetermined posture based on the tracked joint-space configuration of the work manipulator.

18. The system of claim 1, further comprising multiple work manipulators that are each separately moveable from the sensor manipulator, each of the multiple work manipulators configured to:
engage with the workpiece and the other work manipulators, and
generate individual position or function data,
wherein the work controller is configured to receive the individual position or function data for each of the multiple work manipulators and generate an instruction to cause each of the multiple work manipulators to take action to engage with the workpiece based on the received individual position or function data.

19. The system of claim 18, wherein the work controller includes multiple, coordinated work controllers that are each configured to:
receive the individual position or function data for each respective work manipulators of the multiple work manipulators, and
generate an instruction to cause each of the respective multiple work manipulators to take action to engage with the workpiece and the other multiple work manipulators, the instruction based on the received positions or functions data for each of the respective multiple work manipulators.

20. The system of claim 18, wherein the work controller is further configured to generate the instruction to allow the multiple work manipulators to take action to engage with the workpieces and to avoid collision with the other wok manipulators.
